(19) United States
(12) Patent Application Publication
(10) Pub. No.: US 2006/0110354 A1
(43) Pub. Date: May 25, 2006

(54) Concentrated surface-active preparations

(76) Inventors: Joaquin Bigorra Llosas, Sabadell (ES); Nuria Bonastre Gilabert, Barbera del Vall (ES)

Correspondence Address: COGNIS CORPORATION;PATENT DEPARTMENT, 300 BROOKSIDE AVENUE, AMBLER, PA, 19002, US

(21) Appl. No.: 10/532,283

(22) PCT Filed: Oct. 14, 2003
(86) PCT No.: PCT/EP03/11366

§ 371 Date: Sep. 1, 2005

(30) Foreign Application Priority Data
Oct. 23, 2002 (EP) 02023572.7

(51) Int. Cl. C07C 309/15; A61K 8/44

(52) U.S. Cl. 424/70.21; 554/50

(57) ABSTRACT

Concentrated surface-active preparations with low viscosity are disclosed, comprising (a) 25 to 55 wt. % amphoteric or zwitter-ionic surfactants (b) 0.01 to 5 wt. % alkaline sulfates, with the proviso that the amounts are made up to 100 wt % with water and, optionally, further electrolytic salts.

### FIELD OF THE INVENTION

This invention relates generally to surface-active substances and, more particularly, to water-based preparations of amphoteric or zwitterionic surfactants, more particularly betaines, which are distinguished by a low paste viscosity despite a high-active substance concentration.

### PRIOR ART

Amphoteric or zwitterionic surfactants are distinguished by excellent foam and cleaning properties coupled with excellent dermatological compatibility. Accordingly, they are used as important raw materials for the production both of detergents (for example manual dishwashing detergents) and of cosmetic preparations (for example hair shampoos). Within this group of compounds, alkyl betaines and preferably alkyl amidobetaines have acquired particular significance, the most important single product undoubtedly being cocamidopropyl betaine which is commercially available, for example, under the name of Dehyton® PK (Cognis).

Amphoteric or zwitterionic surfactants are marketed as water-based preparations, the interest of the manufacturer being directed towards marketing concentrates, i.e. reducing the quantity of water in the preparations to a minimum. However, there are limits to such a reduction because the active substance content cannot be increased at will because—depending on other ingredients—the surface-active substances begin to form a lamellar gel-form phase above a value of about 40% by weight. The extreme increase in viscosity which this involves and which, occasionally, only occurs during storage makes it virtually impossible to transport or pump the products. However, such products are unsuitable for marketing.

In the past, there has been no shortage of attempts to solve the problem of the increase in viscosity and gelation during storage. Such attempts have included in particular the addition of free fatty acids, optionally together with glycerol, which is proposed in European patent EP 0560114 B1 (Goldschmidt). However, the disadvantage of this is that fatty acids are comparatively expensive additives and are not looked upon favorably in all end products subsequently produced and a measurable reduction in paste viscosity only occurs at values of 1 to 1.5% by weight. Alternatively, it is proposed in European patent EP 0730572 B1 (Cognis) to reduce the viscosity by the addition of hydroxycarboxylic acids or salts thereof, more especially sodium citrate. Although smaller quantities are sufficient in this case, the price—which is still above that of fatty acids—makes the process relatively unattractive.

Accordingly, the problem addressed by the present invention was to provide new concentrated, water-based amphoteric or zwitterionic surfactant preparations which would be distinguished by a low Brookfield viscosity (20° C., spindle 1, 10 r.p.m.) of, in particular, less than 5,000 mPas for an active substance concentration of, for example, 25 to 55% by weight and which would not gel in storage.

### DESCRIPTION OF THE INVENTION

The present invention relates to concentrated, low-viscosity surface-active preparations containing
(a) 25 to 50% by weight of amphoteric or zwitterionic surfactants and
(b) 0.01 to 5% by weight of alkali metal sulfates,
   with the proviso that the quantities add up to 100% by weight with water and optionally other electrolyte salts.

It has surprisingly been found that even the presence of very small quantities of alkali metal sulfates, more especially sodium sulfate, is sufficient clearly to reduce the paste viscosity of highly concentrated water-based preparations of amphoteric or zwitterionic surfactants both by addition during production and by subsequent addition to the pastes. More particularly, even the unwanted effect of gradual gelation is reliably prevented.

Amphoteric Surfactants

Examples of suitable amphoteric or zwitterionic surfactants are alkyl betaines, alkyl amidobetaines, aminopropionates, aminoglycinates, imidazolinium betaines and sulfobetaines. Examples of suitable alkyl betaines are the carboxyalkylation products of secondary and, in particular, tertiary amines corresponding to formula (I): 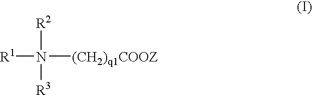
in which R¹ represents alkyl and/or alkenyl groups containing 6 to 22 carbon atoms, R² represents hydrogen or alkyl groups containing 1 to 4 carbon atoms, R³ represents alkyl groups containing 1 to 4 carbon atoms, q1 is a number of 1 to 6 and Z is an alkali metal and/or alkaline earth metal or ammonium. Typical examples are the carboxymethylation products of hexylmethyl amine, hexyldimethyl amine, octyldimethyl amine, decyldimethyl amine, dodecylmethyl amine, dodecyldimethyl amine, dodecylethylmethyl amine, C_{12/14} cocoalkyldimethyl amine, myristyldimethyl amine, cetyldimethyl amine, stearyldimethyl amine, stearylethylmethyl amine, oleyldimethyl amine, C_{16/18} tallowalkyl dimethyl amine and technical mixtures thereof.

Also suitable are carboxyalkylation products of amidoamines corresponding to formula (II): 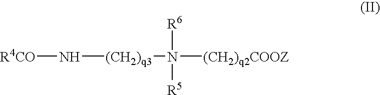
in which R⁴CO is an aliphatic acyl group containing 6 to 22 carbon atoms and 0 or 1 to 3 double bonds, R⁵ is hydrogen or represents alkyl groups containing 1 to 4 carbon atoms, R⁶ represents alkyl groups containing 1 to 4 carbon atoms, q2 is a number of 1 to 6, q3 is a number of 1 to 3 and Z is again an alkali metal and/or alkaline earth metal or ammonium. Typical examples are reaction products of fatty acids containing 6 to 22 carbon atoms, namely caproic acid, caprylic acid, capric acid, lauric acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid and technical mixtures thereof, with N,N-dimethylaminoethyl amine, N,N-dimethylaminopropyl amine, N,N-diethylaminoethyl amine and N,N-diethylaminopropyl amine which are condensed with sodium chloroacetate. A condensation product of C_{8/18}-cocofatty acid-N,N-dimethylaminopropyl amide with sodium chloroacetate is preferably used.

Imidazolinium betaines may also be used. These compounds are also known compounds which may be obtained, for example, by cyclizing condensation of 1 or 2 mol fatty acid with polyfunctional amines such as, for example, aminoethyl ethanolamine, (AEEA) or diethylenetriamine. The corresponding carboxyalkylation products are mixtures of different open-chain betaines. Typical examples are condensation products of the fatty acids mentioned above with AEEA, preferably imidazolines based on lauric acid or—again—C _{12/14} cocofatty acid which are subsequently betainized with sodium chloroacetate.

Alkali Metal Sulfates

The alkali metal sulfates are typical inorganic salts such as, for example, potassium or sodium sulfate and mixtures thereof. The use of sodium sulfate is preferred for reasons of price and availability.

Preparations

Surface-active preparations in the context of the present invention are understood to be the water-containing pastes of the amphoteric or zwitterionic surfactants as such. More particularly, they contain no other surface-active substances, but—from their production—electrolyte salts, more particularly sodium chloride, unreacted starting materials and optionally small quantities of free fatty acids or salts thereof. The preparations may be alkaline or acidic, i.e. they have a pH of typically 6 to 9 or 1 to 6. The content of amphoteric or zwitterionic surfactants, which is referred to as the “active substance content”, may be in the range from 25 to 55% by weight, based on the preparation, and is generally in the range from 35 to 45% by weight. By contrast, the content of alkali metal sulfate may be between 0.01 and 5% by weight and is preferably between 0.1 and 3% by weight and more particularly between 0.5 and 1% by weight, again based on the preparation. The quantity of other ingredients (which do not contribute to the surface-active character of the preparation), more especially electrolyte salts, such as sodium chloride, is typically between 5 and 10% by weight. This results in a typical water content of ca. 35 to ca. 60% by weight and, more particularly, 40 to 50% by weight. The Brookfield viscosity of such preparations (as measured in an RVT viscosimeter at 20° C., spindle 1, 10 r.p.m.) is less than 5,000 mPas and preferably in the range from 1,000 to 2,500 mPas.

Production Process

A particular advantage of the present invention is that the alkali metal sulfates may be added both during and after production. Addition after production may be preferred in cases where the paste viscosity is to be adjusted to a very precise value. Accordingly, the present invention also relates to a process for the production of concentrated, low-viscosity surface-active preparations which is characterized in that fatty amines or fatty acid amidoamines are betainized with halocarboxylic acids or alkali metal salts thereof in known manner in the presence of alkali metal sulfates. So far as the betainization reaction is concerned, reference is specifically made to the disclosures of the prior-art publications cited above, so that there is no need for them to be repeated at length here. Alternatively, the present invention also relates to a second process for the production of concentrated, low-viscosity surface-active preparations which is characterized in that alkali metal sulfates are added to water-containing pastes of alkyl betaines and/or alkyl amidobetaines. As explained above, the quantity of alkali metal sulfates is calculated so that their content in the final preparations is from 0.01 to 5% by weight, preferably from 0.1 to 3% by weight and more particularly from 0.5 to 1% by weight.

Commercial Applications

The addition of alkali metal sulfates, more especially sodium sulfate, even in very small quantities, is particularly suitable for reducing the viscosity of highly concentrated, for example—based on active substance—35 to 45% by weight alkyl betaine or alkyl amidobetaine pastes to such an extent that they can be pumped and transported without difficulty. It does not matter whether the alkali metal sulfates are added during or after the production process. Accordingly, the present invention also relates to the use of alkali metal sulfates for reducing the viscosity of concentrated aqueous preparations of amphoteric or zwitterionic surfactants, the quantity added—based on the preparations—being from 0.01 to 5% by weight, preferably from 0.1 to 3% by weight and more particularly from 0.5 to 1% by weight. The concentrates thus obtained are suitable, for example, for the production of manual dishwashing detergents or hair shampoos in which they may be present, for example, in quantities of 1 to 15% by weight and more particularly 3 to 8% by weight.

### EXAMPLES

### Comparison Example 1

1083 g (5.3 mol) of a hydrogenated cocofatty acid and 552 g (5.4 mol) of N,N-dimethyl aminopropylamine were introduced into a 2-liter four-necked flask equipped with a thermometer, condenser, water separator, nitrogen inlet pipe and stirrer and heated under nitrogen to 180° C. The water of condensation was continuously removed from the reaction vessel. The reaction was continued until the acid value of the mixture had fallen below 5. Excess amine was then removed in vacuo. 1500 g of cocofatty acid amidopropyl dimethylamine were obtained in this way and were adjusted with water to an active-substance concentration of 45% by weight. The preparation was virtually solid.

### Example 1

Comparison Example 1 was repeated, except that the reaction of the cocofatty acid with the N,N-dimethyl aminopropylamine was carried out in the presence of 7.5 g (corresponding to 0.5% by weight, based on the final composition) of sodium sulfate. The mixture was again adjusted with water to an active substance concentration of 45% by weight and had a Brookfield viscosity (20° C., spindle 1, 10 r.p.m.) of 4,500 mPas.

### Examples 2 and 3, Comparison Example C2

Quantities of 0.5% by weight and 1% by weight of sodium sulfate were subsequently added to an aqueous preparation of cocamidopropyl betaine (Dehyton® PK, Cognis) with an active substance concentration of 45% by weight. The appearance and the viscosity of the pastes were then determined at 5, 10, 15 and 25° C. The results are set out in Table 1.

TABLE 1
Appearance and viscosity of cocamidopropyl betaine pastes

| Quantity of sodium sulfate added [% by weight] | | |
|---|---|---|
| None | 0.5 | 1.0 |

Appearance

| | | | |
|---|---|---|---|
| at 25° C. | Gelled | Thinly liquid, cloudy | Thinly liquid, clear |
| at 15° C. | Gelled | Liquid, cloudy | Thinly liquid, clear |
| at 10° C. | Solid | Viscous | Thinly liquid, clear |
| at 5° C. | Solid | Solid | Thinly liquid, clear |

Viscosity [mPas]

| | | |
|---|---|---|
| at 25° C. | Gelled | <3,000 |
| at 15° C. | Gelled | <3,000 |
| at 10° C. | Solid | <3,000 |
| at 5° C. | Solid | <3,000 |

1-11. (canceled)

12. A concentrated, low-viscosity surface-active preparation, comprising
(a) 25 to 50% by weight of amphoteric or zwitterionic surfactants and
(b) 0.01 to 5% by weight of at least one alkali metal sulfate,
with the proviso that the quantities add up to 100% by weight with water and optionally other electrolyte salts.

13. The preparation according to claim 12, wherein the amphoteric or zwitterionic surfactants are selected from the group consisting of alkyl betaines, alkyl amidobetaines, aminopropionates, aminoglycinates, imidazolinium betaines and sulfobetaines.

14. The preparation according to claim 13, wherein alkyl betaines have the formula (I): 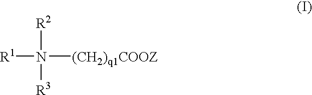
in which R¹ is an alkyl or alkenyl group containing 6 to 22 carbon atoms, R² is hydrogen or an alkyl group containing 1 to 4 carbon atoms, R³ is an alkyl group containing 1 to 4 carbon atoms, q1 is a number of 1 to 6, and Z is an alkali metal, an alkaline earth metal or ammonium.

15. The preparation according to claim 13, wherein the alkyl amidobetaines have the formula (II): 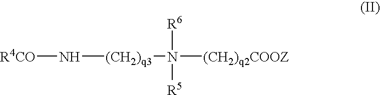
in which R⁴CO is an aliphatic acyl group containing 6 to 22 carbon atoms and 0 or 1 to 3 double bonds, R⁵ is hydrogen or an alkyl group containing 1 to 4 carbon atoms, R⁶ is an alkyl group containing 1 to 4 carbon atoms, q2 is a number of 1 to 6, q3 is a number of 1 to 3, and Z is an alkali metal, an alkaline earth metal or ammonium.

16. The preparation according to claim 12, wherein the at least one alkali metal sulfate is selected from sodium sulfate, potassium sulfate and mixtures thereof.

17. The preparation according to claim 12, wherein the at least one alkali metal sulfate is sodium sulfate.

18. The preparation according to claim 12, wherein the at least one alkali metal sulfate is present in the amount of 0.1 to 3% by weight.

19. The preparation according to claim 12, having a Brookfield viscosity, as measured in an RVT viscosimeter (20° C., spindle 1, 10 r.p.m.), of less than 5,000 mPas.

20. The preparation according to claim 12, having a Brookfield viscosity, as measured in an RVT viscosimeter (20° C., spindle 1, 10 r.p.m.), of 1,000 to 2,500 mPas.

21. A process for the production of a concentrated, low-viscosity, surface-active preparation, said process comprising forming one or more amphoteric or zwitterionic surfactants by betainizing at least one fatty amine or fatty acid amidoamide with at least one halocarboxylic acid or alkali metal salt thereof in the presence of at least one alkali metal sulfate.

22. The process according to claim 21, wherein the resulting low-viscosity surface-active preparation comprises
(a) 25 to 50% by weight of one or more amphoteric or zwitterionic surfactants and
(b) 0.01 to 5% by weight of at least one alkali metal sulfate,
with the proviso that the quantities add up to 100% by weight with water and optionally other electrolyte salts.

23. The process according to claim 21, wherein, wherein the resulting one or more amphoteric or zwitterionic surfactants are selected from the group consisting of alkyl betaines, alkyl amidobetaines, aminopropionates, aminoglycinates, imidazolinium betaines and sulfobetaines.

24. The process according to claim 21, wherein the at least one alkali metal sulfate is selected from sodium sulfate, potassium sulfate and mixtures thereof.

25. The process according to claim 22, wherein the at least one alkali metal sulfate is present in the amount of 0.1 to 3% by weight.

26. The process according to claim 21, wherein the resulting low-viscosity surface-active preparation has a Brookfield viscosity, as measured in an RVT viscosimeter (20° C., spindle 1, 10 r.p.m.), of less than 5,000 mPas.

27. A process for reducing the viscosity of a water-containing paste of alkyl betaines and/or alkyl amidobetaines having a Brookfield viscosity, as measured in an RVT viscosimeter (20° C., spindle 1, 10 r.p.m.), of greater than 5,000 mPas, said process comprising adding at least one alkali metal sulfate to the water-containing paste.

28. The process according to claim 27, wherein the resulting reduced-viscosity, water-containing paste comprises
(a) 25 to 50% by weight of alkyl betaines and/or alkyl amidobetaines and
(b) 0.01 to 5% by weight of at least one alkali metal sulfate,
with the proviso that the quantities add up to 100% by weight with water and optionally other electrolyte salts.

29. The process according to claim 27, wherein the alkyl betaines and/or alkyl amidobetaines are selected from the group consisting of alkyl betaines, alkyl amidobetaines, aminopropionates, aminoglycinates, imidazolinium betaines and sulfobetaines.

30. The process according to claim 27, wherein the at least one alkali metal sulfate is selected from sodium sulfate, potassium sulfate and mixtures thereof.

31. The process according to claim 28, wherein the at least one alkali metal sulfate is present in the amount of 0.1 to 3% by weight.

32. The process according to claim 27, wherein the resulting reduced-viscosity, water-containing paste has a Brookfield viscosity, as measured in an RVT viscosimeter (20° C., spindle 1, 10 r.p.m.), of 1,000 to 2,500 mPas.
